(19) United States
(12) Patent Application Publication
(10) Pub. No.: US 2013/0295148 A1
(43) Pub. Date: Nov. 7, 2013

(54) SOLID COSMETIC MAKEUP AND/OR CARE COMPOSITION

(71) Applicants: Coralie Claude-Foly, Verneuil en Halatte (FR); Xavier Blin, Paris (FR); Françoise Blondeau, Choisy Le Roi (FR); Zohra Moujahed, Savigny-Sur-Orge (FR); Eric Quemin, Saint Remy Les Chevreuse (FR)

(72) Inventors: Coralie Claude-Foly, Verneuil en Halatte (FR); Xavier Blin, Paris (FR); Françoise Blondeau, Choisy Le Roi (FR); Zohra Moujahed, Savigny-Sur-Orge (FR); Eric Quemin, Saint Remy Les Chevreuse (FR)

(73) Assignee: L'OREAL, Paris (FR)

(21) Appl. No.: 13/885,567

(22) PCT Filed: Nov. 10, 2011
(86) PCT No.: PCT/IB2011/055031

§ 371 Date: Jun. 28, 2013

Related U.S. Application Data
(60) Provisional application No. 61/417,082, filed on Nov. 24, 2010.

(30) Foreign Application Priority Data
Nov. 15, 2010 (FR) 10 59376

(51) Int. Cl. A61K 8/02; A61Q 1/12; A61Q 1/02

(52) U.S. Cl. CPC A61K 8/022; A61Q 1/02; A61Q 1/12
USPC 424/401; 424/69; 424/63

(57) ABSTRACT

The present invention relates to a solid cosmetic makeup and/or care composition comprising: —at least 30% by weight of a pulverulent phase relative to its total weight, and—at least 15% by weight of non-volatile oil(s) relative to its total weight, said composition being in the form of a powder. It also targets a corresponding makeup and/or care method.

The present invention targets the field of cosmetic care and/or makeup compositions, which are completely or partly in the form of particles or are also referred to as pulverulent.

The galenic forms conventionally adopted for solid compositions are generally loose or pressed powders. These galenic forms are particularly appreciated by users with regard to their lightness, softness, tack-free aspect or non-greasy feel.

As non-limiting illustrations of the pulverulent galenic forms more particularly considered in the field of makeup, mention may be made especially of loose or compact foundation powders, face powders or eyeshadows. The function of the above-mentioned powders is mainly to give colour, mattness and even, for those more particularly intended for facial skin, to improve the wear property of a foundation or, if used alone, to give, in addition, coverage (foundation powder).

However, these powder compositions have specifically, with regard to their lightness, a tendency to be removed in the course of the day, and it is therefore generally necessary for the user to touch up the makeup in the course of the day.

Furthermore, with regard to their low content of comfort oils, that is to say non-volatile oils, these powders have the drawback of being uncomfortable since they are drying. This discomfort is due to the fact that it is impossible to introduce a high percentage of non-volatile oils into the solid composition if it is desired in addition to retain a loose powder aspect or a non-greasy aspect thereof. Indeed, conventional methods do not allow the incorporation of a binder beyond an amount of around 10% on average in a pulverulent phase.

Consequently, to date there remains a need for cosmetic compositions that meet the expectations of consumers both in sensory terms, that is to say lightness and softness to the touch and in terms of comfort, that is to say free of a feeling of discomfort but on the contrary capable of providing a moisturizing or even fresh feeling.

The objective of the present invention is, precisely, to satisfy this need.

Thus, the inventors have, unexpectedly, observed that it is possible to formulate care and/or makeup compositions in an original galenic form, without however being detrimental to their properties, but moreover by improving their organoleptic properties, especially in terms of softness, comfort on wearing or application, and homogeneity.

Thus, the present invention relates, according to one of its aspects, to a solid cosmetic makeup and/or care composition, especially for the skin and/or lips comprising:
at least 30% by weight of a pulverulent phase relative to its total weight, and
at least 15% by weight of non-volatile oil(s) relative to its total weight, said composition being in the form of a powder.

Due to the presence, in a proportion of at least 15% by weight, of non-volatile oils, the composition imparts to the user significant comfort, a pleasant sensory feeling, softness, an emollient property and possesses better adhesion to the keratin material, especially the skin.

Preferably, the composition is an eyeshadow, a composition for the eyebrows, an eyeliner, a care and/or makeup product for the lips (e.g. lipstick, lipgloss), a blusher, or a powder that can be applied to the face, in particular a foundation powder. Even more preferentially, the composition is a foundation, an eyeshadow, a blusher or a product for the lips.

According to another of its aspects, the present invention relates to a method for making up and/or caring for keratin materials and especially the skin and lips, and more particularly the skin of the face, in which a composition as defined above is applied to said keratin materials.

Solid Composition

The composition according to the invention is solid.

The term “solid” characterizes the state of the composition at room temperature (25° C.) and at atmospheric pressure (760 mmHg), i.e. a composition of high consistency, which retains its form during storage. As opposed to “fluid” compositions, it does not flow under its own weight. It is advantageously characterized by a hardness as defined below.

More specifically, it is in the form of a loose or pressed powder.

It is preferentially a pressed powder.

The compositions according to the invention may be characterized by indentation measurements (compressive force or penetration force) in order to evaluate their hardness.

The term “hardness” is understood to mean the maximum penetration force obtained during the operation described below and expressed in newtons.

It is measured at 20° C. using a texture analyser sold under the name TAXT2i by the company RHEO, equipped with a stainless-steel spindle in the form of a bead 5 mm in diameter by measuring the change in force (compressive force or penetration force) (F) as a function of time, during the following operation:
A sample of the composition to be characterized is pressed at 800 kg/cm² in a small dish having a thickness at least equal to 10 mm and a surface area at least equal to 3 cm². The sample to be measured should have, once pressed, a minimum thickness of 3 mm and should not have any visible defect: cracks, large fragments, areas of crumbling. The sample is thermostatically controlled at 20° C.
The spindle is displaced at a speed of 0.1 mm/s and then penetrates into the product to a penetration depth of 300 μm. When the spindle has penetrated into the product to a depth of 300 μm, the spindle is removed at a speed of 0.1 mm/s. During the withdrawal of the spindle, the force (compressive force) decreases greatly until it becomes 0 after a time t. During the operation, the spindle moves back and forth in 6 seconds.

Nine measurements are taken for one and the same composition, either at different locations evenly distributed and spaced out over the sample, or on different samples for one and the same composition. The average of these nine measurements indicates the hardness of the composition with a 95% confidence interval.

Thus, the compositions according to the invention may advantageously have a hardness that varies from 0.01 to 5 newtons and especially from 0.04 to 2 N and more particularly of the order from 0.07 to 1 N, and more particularly still from 0.09 to 0.3 N.

A cosmetic composition according to the invention comprises at least one fatty phase as binder.

Binder Fatty Phase

The non-volatile oil(s) required according to the invention may form all or part of the fatty phase, also referred to as binder, of said composition.

The term “oil” means a water-immiscible non-aqueous compound that is liquid at room temperature (25° C.) and at atmospheric pressure (760 mmHg).

This liquid fatty phase is advantageously present in a proportion of at least 15% by weight relative to the total weight of said composition, and preferably in a proportion from 15% to 60%, in particular from 25% to 45% and preferentially from 30% to 45% by weight relative to the total weight of the composition.

Non-Volatile Oils

The term “non-volatile oil” means an oil that remains on the skin or the keratin fibre at room temperature and atmospheric pressure. More specifically, a non-volatile oil has an evaporation rate strictly less than 0.01 mg/cm²/min.

To measure this evaporation rate, 15 g of oil or oil mixture to be tested are placed in a crystallizing dish 7 cm in diameter, placed on a balance that is in a large chamber of about 0.3 m³ which is temperature-regulated, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing said oil or said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit of area (cm²) and per unit of time (minutes).

The non-volatile oils suitable for the invention may be chosen especially from non-volatile hydrocarbon-based, fluoro and/or silicone oils.

According to one particular embodiment, the composition comprises at least one non-volatile hydrocarbon-based oil.

According to another embodiment, the composition comprises at least one non-volatile silicone oil.

Non-volatile oils that may especially be mentioned include:
hydrocarbon-based oils of animal origin, such as perhydrosqualene,
hydrocarbon-based oils of plant origin, such as phytostearyl esters, such as phytostearyl oleate, phytostearyl isostearate and lauroyl/octyldodecyl/phytostearyl glutamate (Ajinomoto, Eldew PS203), triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, winter squash oil, millet oil, barley oil, quinoa oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camelina oil, canola oil, carrot oil, safflower oil, flax oil, rapeseed oil, cottonseed oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant seed oil, kiwi seed oil, grapeseed oil, pistachio oil, winter squash oil, pumpkin oil, quinoa oil, musk rose oil, sesame oil, soybean oil, sunflower oil, castor oil and watermelon oil, and mixtures thereof, or alternatively caprylic/capric acid triglycerides, such as those sold by the company Stearineries Dubois or those sold under the names Miglyol 810®, 812® and 818® by the company Dynamit Nobel,
linear or branched hydrocarbons of mineral or synthetic origin, such as liquid paraffins and derivatives thereof, petroleum jelly, polydecenes, polybutenes, hydrogenated polyisobutene such as Parleam, and squalane;
synthetic ethers containing from 10 to 40 carbon atoms;
synthetic esters, for instance oils of formula R₁COOR₂, in which R₁ represents a linear or branched fatty acid residue containing from 1 to 40 carbon atoms, and R₂ represents a hydrocarbon-based chain that is especially branched, containing from 1 to 40 carbon atoms provided that R₁+R₂≧10. The esters may be chosen especially from fatty acid esters of alcohols, for instance cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoates, polyethylene glycol diheptanoate, propylene glycol 2-diethylhexanoate, and mixtures thereof, C₁₂-C₁₅ alcohol benzoates, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate;
polyol esters and pentaerythritol esters, for instance dipentaerythrityl tetrahydroxystearate/tetraisostearate,
esters of diol dimers and of diacid dimers, such as Lusplan DD-DA5® and Lusplan DD-DA7® sold by the company Nippon Fine Chemical and described in patent application US 2004-175338,
copolymers of a diol dimer and of a diacid dimer and esters thereof, such as dilinoleyl diol dimer/dilinoleic dimer copolymers and esters thereof, for instance Plandool-G,
copolymers of polyols and of diacid dimers, and esters thereof, such as Hailuscent ISDA or the dilinoleic acid/butanediol copolymer,
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol,
C₁₂-C₂₂ higher fatty acids, such as oleic acid, linoleic acid or linolenic acid, and mixtures thereof, and
dialkyl carbonates, the two alkyl chains possibly being identical or different, such as dicaprylyl carbonate sold under the name Cetiol CC®, by Cognis,
oils of high molar mass, in particular having a molar mass ranging from about 400 to about 10 000 g/mol, in particular from about 650 to about 10 000 g/mol, in particular from about 750 to about 7500 g/mol and more particularly ranging from about 1000 to about 5000 g/mol. As oils of high molar mass that may be used in the present invention, mention may especially be made of oils chosen from:
   lipophilic polymers,
   linear fatty acid esters with a total carbon number ranging from 35 to 70,
   hydroxylated esters,
   aromatic esters,
   C₂₄-C₂₈ branched fatty acid or fatty alcohol esters,
   silicone oils,
   oils of plant origin,
   and mixtures thereof
fluoro oils that are optionally partially hydrocarbon-based and/or silicone-based, for instance fluorosilicone oils, fluoropolyethers and fluorosilicones as described in document EP-A-847 752;
silicone oils, for instance linear or cyclic non-volatile polydimethylsiloxanes (PDMSs); polydimethylsiloxanes comprising alkyl, alkoxy or phenyl groups, which are pendant or at the end of a silicone chain, these groups containing from 2 to 24 carbon atoms; phenyl silicones, for instance phenyl trimethicones, phenyl dimethicones, phenyl-trimethylsiloxydiphenylsiloxanes, diphenyl dimethicones, diphenylmethyldiphenyl-trisiloxanes and 2-phenylethyltrimethylsiloxysilicates, and
mixtures thereof.

According to one preferred variant, the non-volatile oils required according to the invention are chosen from silicone oils.

Thus, a composition according to the invention may comprise at least one non-volatile silicone oil.

According to one particular embodiment, the composition comprises at least one non-volatile silicone oil having a viscosity less than or equal to 1000 cst at 25° C.

According to another embodiment, the composition comprises at least one non-volatile hydrocarbon-based oil chosen from the oils of formula R₁COOR₂, in which R₁ represents a linear or branched fatty acid residue containing from 1 to 40 carbon atoms and R₂ represents a hydrocarbon-based chain, which is especially branched, containing from 1 to 40 carbon atoms, provided that R₁+R₂≧10, in particular isononanoic acid esters and preferably isononyl isononanoate.

Advantageously, a composition according to the invention comprises a content of non-volatile oil(s) ranging from 20% to 100% by weight, preferably ranging from 40% to 95% by weight, and preferentially ranging from 50% to 90% by weight, relative to the total weight of the liquid fatty phase.

More particularly, the content of non-volatile oil(s) ranges from 15% to 60% by weight and in particular from 30% to 45% by weight relative to the total weight of the composition.

According to one preferred embodiment, the non-volatile silicone oil(s) represent(s) more than 50% by weight of the total non-volatile oils, in particular more than 70% by weight, preferably more than 80%, or even more than 90% by weight of the total non-volatile oils.

Thus, a composition of the invention could comprise a content of non-volatile silicone oils ranging from 15% to 60% by weight and in particular from 30% to 45% by weight relative to the total weight of the composition.

Besides the non-volatile oil required according to the invention, the composition according to the invention may comprise from 0 to 20% by weight and especially from 0.1% to 20% by weight of additional oils, i.e. volatile oils, relative to the total weight of the composition.

The term “volatile oil” means any non-aqueous medium that is capable of evaporating on contact with the skin or the lips in less than one hour, at room temperature and atmospheric pressure. The volatile oil is a cosmetic volatile oil, which is liquid at room temperature. More specifically, a volatile oil has an evaporation rate of between 0.01 and 200 mg/cm²/min, limits included. The oil may be a volatile oil, especially chosen from volatile silicone oils or volatile non-silicone oils.

As volatile oils that can be used in the invention, use may be made of volatile non-silicone oils, especially C₈-C₁₆ isoparaffins for instance isododecane, isodecane, isohexadecane and, for example, the oils sold under the trade names Isopar, Permethyl and especially isododecane (Permethyl 99 A).

As volatile silicone oils that can be used in the invention, mentioned may be made of linear or cyclic silicones containing from 2 to 7 silicon atoms, these silicones optionally comprising alkyl or alkoxy groups containing from 1 to 10 carbon atoms. In particular, mention may be made of octamethylcyclotetrasiloxane, decamethylcyclopentasiloxane, dodecamethylcyclohexasiloxane, heptamethylhexyl-trisiloxane, heptamethyloctyltrisiloxane, octamethyltrisiloxane and decamethyl-tetrasiloxane, and mixtures thereof.

A composition of the invention may comprise less than 2% by weight, or even less than 1% by weight of volatile oil or else is completely free of volatile oil.

Structuring Agents of the Liquid Fatty Phase

A composition according to the invention may also comprise a structuring agent of the liquid fatty phase chosen from a wax, a silicone resin, a pasty compound, and mixtures thereof.

According to one preferred embodiment, the composition according to the invention comprises at least one wax.

The wax under consideration in the context of the present invention is generally a lipophilic compound that is solid at room temperature (25° C.), with a solid/liquid reversible change of state, having a melting point of greater than or equal to 30° C., which may be up to 200° C. and in particular up to 120° C.

In particular, the waxes that are suitable for the invention may have a melting point of greater than or equal to 45° C. and in particular greater than or equal to 55° C.

The waxes that may be used in the compositions according to the invention are chosen from waxes that are solid at room temperature of animal, plant, mineral or synthetic origin, and mixtures thereof.

As illustrations of waxes that are suitable for the invention, mention may be made especially of hydrocarbon-based waxes, for instance beeswax, lanolin wax, Chinese insect waxes, rice bran wax, carnauba wax, candelilla wax, ouricury wax, esparto grass wax, berry wax, shellac wax, Japan wax and sumach wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane)tetrastearate sold under the name Hest 2T-4S® by the company Heterene.

Mention may also be made of silicone waxes (C₃₀₋₄₅ alkyl dimethicone) and fluoro waxes.

The waxes obtained by hydrogenation of castor oil esterified with cetyl alcohol, sold under the names Phytowax ricin 16L64® and 22L73® by the company Sophim, may also be used. Such waxes are described in patent application FR-A-2 792 190.

A wax that may be used is a C₂₀-C₄₀ alkyl (hydroxystearyloxy)stearate (the alkyl group containing from 20 to 40 carbon atoms), alone or as a mixture.

Such a wax is especially sold under the names Kester Wax K 82 P®, Hydroxypolyester K 82 P® and Kester Wax K 80 P® by the company Koster Keunen.

As microwaxes that may be used in the compositions according to the invention, mention may be made especially of carnauba microwaxes, such as the product sold under the name MicroCare 350® by the company Micro Powders, synthetic-wax microwaxes, such as the product sold under the name MicroEase 1145® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and of synthetic wax, such as the product sold under the name Micro Care 325® by the company Micro Powders, polyethylene microwaxes, such as the products sold under the names Micropoly 200®, 220®, 220L® and 2505® by the company Micro Powders, and polytetrafluoroethylene microwaxes, such as the products sold under the names Microslip 519® and 519 L® by the company Micro Powders.

According to one particular embodiment, the composition comprises at least carnauba microwaxes, such as the products sold under the name MicroCare 350® by the company Micro Powders.

In the case of microwaxes, they are generally constituents of the pulverulent phase, as described below, and act as a structuring agent for this phase.

The composition according to the invention may comprise a content of waxes or microwaxes ranging from 0 to 20% by weight relative to the total weight of the composition; it may in particular contain from 1% to 10% by weight relative to the total weight of the composition.

Likewise, a composition according to the invention may contain a silicone resin.

Examples of these silicone resins that may be mentioned include:

siloxysilicates, which may be trimethylsiloxysilicates of formula [(CH₃)₃SiO]ₓ(SiO_{4/2})_{y} (MQ units) in which x and y are integers ranging from 50 to 80,

polysilsesquioxanes of formula (CH₃SiO_{3/2})ₓ (T units) in which x is greater than 100 and at least one of the methyl radicals of which may be substituted with a group R as defined above,

polymethylsilsesquioxanes, which are polysilsesquioxanes in which none of the methyl radicals is substituted with another group. Such polymethylsilsesquioxanes are described in document U.S. Pat. No. 5,246,694, the content of which is incorporated by reference.

As examples of commercially available polymethylsilsesquioxane resins, mention may be made of those sold:

by the company Wacker under the reference Resin MK, such as Belsil PMS MK: polymer comprising CH₃SiO_{3/2} repeating units (T units), which may also comprise up to 1% by weight of (CH₃)₂SiO₂₁₂ units (D units) and having an average molecular weight of about 10 000, or

by the company Shin-Etsu under the references KR-220L, which are composed of T units of formula CH₃SiO_{3/2} and contain Si—OH (silanol) end groups, under the reference KR-242A, which comprise 98% of T units and 2% of dimethyl D units and contain Si—OH end groups, or also under the reference KR-251, comprising 88% of T units and 12% of dimethyl D units and contain Si—OH end groups.

Siloxysilicate resins that may be mentioned include trimethylsiloxysilicate (TMS) resins optionally in the form of powders. Such resins are sold under the reference SR1000 by the company Momentive Performance Materials or under the reference TMS 803 by the company Wacker. Mention may also be made of trimethylsiloxysilicate resins sold in a solvent such as cyclomethicone, sold under the name KF-7312J by the company Shin-Etsu or DC 749 and DC 593 by the company Dow Corning.

Advantageously, the silicone resin, for instance the trimethylsiloxysilicate resin, is present in a content ranging from 0.5% to 30%, or better still from 1% to 25% or even better still from 5% to 25% relative to the total weight of the composition.

The expressions “pasty compounds” or “pasty fatty compounds” are especially intended to denote a lipophilic fatty compound that undergoes a reversible solid/liquid change of state, exhibiting anisotropic crystal organization in the solid state, and that comprises, at a temperature of 23° C., a liquid fraction and a solid fraction.

The pasty compound is advantageously chosen from:

lanolin and its derivatives,

polyol ethers chosen from polyalkylene glycol pentaerythrityl ethers, fatty alcohol ethers of sugar, and mixtures thereof, polyethylene glycol pentaerythrityl ether comprising five oxyethylene (5 OE) units (CTFA name: PEG-5 Pentaerythrityl Ether), polypropylene glycol pentaerythrityl ether comprising five oxypropylene (5 OP) units (CTFA name: PPG-5 Pentaerythrityl Ether) and mixtures thereof, and more especially the mixture PEG-5

Pentaerythrityl Ether, PPG-5 Pentaerythrityl Ether and soybean oil, sold under the name Lanolide by the company Vevy, which is a mixture in which the constituents are in a 46/46/8 weight ratio: 46% PEG-5 Pentaerythrityl Ether, 46% PPG-5 Pentaerythrityl Ether and 8% soybean oil;

polymeric or non-polymeric silicone compounds

polymeric or non-polymeric fluoro compounds

vinyl polymers, especially:
olefin homopolymers and copolymers,
hydrogenated diene homopolymers and copolymers,
linear or branched oligomers, homopolymers or copolymers of alkyl (meth)acrylates preferably containing a C₈-C₃₀ alkyl group,
oligomers, homopolymers and copolymers of vinyl esters containing C₈-C₃₀ alkyl groups,
oligomers, homopolymers and copolymers of vinyl ethers containing C₈-C₃₀ alkyl groups,

liposoluble polyethers resulting from the polyetherification between one or more C₂-C₁₀₀ and preferably C₂-C₅₀ diols,

esters,

and/or mixtures thereof.

Among the liposoluble polyethers that are particularly preferred are copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30. In this family, mention will be made especially of copolymers such that the long-chain alkylene oxides are arranged in blocks having an average molecular weight from 1000 to 10 000, for example a polyoxyethylene/polydodecyl glycol block copolymer such as the ethers of dodecanediol (22 mol) and of polyethylene glycol (45 OE) sold under the brand name Elfacos ST9® by Akzo Nobel.

Among the esters, the following are especially preferred:

esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, especially such as those sold under the brand name Softisan 649® by the company Sasol,

the arachidyl propionate sold under the brand name Waxenol 801 by Alzo,

phytosterol esters,

fatty acid triglycerides and derivatives thereof,

pentaerythritol esters,

non-crosslinked polyesters resulting from polycondensation between a linear or branched C₄-C₅₀ dicarboxylic acid or polycarboxylic acid and a C₂-C₅₀ diol or polyol,

aliphatic esters of an ester resulting from the esterification of an aliphatic hydroxycarboxylic acid ester with an aliphatic carboxylic acid. Preferably, the aliphatic carboxylic acid comprises from 4 to 30 and preferably from 8 to 30 carbon atoms. It is preferably chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid and docosanoic acid, and mixtures thereof. The aliphatic carboxylic acid is preferably branched. The aliphatic hydroxycarboxylic acid ester is advantageously derived from a hydroxylated aliphatic carboxylic acid containing from 2 to 40 carbon atoms, preferably from 10 to 34 carbon atoms and better still from 12 to 28 carbon atoms, and from 1 to 20 hydroxyl groups, preferably from 1 to 10 hydroxyl groups and better still from 1 to 6 hydroxyl groups. The aliphatic hydroxycarboxylic acid ester is chosen from:
a) partial or total esters of saturated linear mono-hydroxylated aliphatic monocarboxylic acids;
b) partial or total esters of unsaturated monohydroxylated aliphatic monocarboxylic acids;
c) partial or total esters of saturated monohydroxylated aliphatic polycarboxylic acids;
d) partial or total esters of saturated polyhydroxylated aliphatic polycarboxylic acids;
e) partial or total esters of C₂ to C₁₆ aliphatic polyols that have reacted with a monohydroxylated or polyhydroxylated aliphatic monocarboxylic or polycarboxylic acid,
   and mixtures thereof,
esters of a diol dimer and of a diacid dimer, where appropriate esterified on their free alcohol or acid function(s) with acid or alcohol radicals, especially dimer dilinoleate esters; such esters may be chosen especially from the esters having the following INCI nomenclature: bis-behenyl/isostearyl/phytosteryl dimer dilinoleyl dimer dilinoleate (Plandool G®), phytosteryl isostearyl dimer dilinoleate (Lusplan PI-DA®, Lusplan PHY/IS-DA®), and mixtures thereof,
hydrogenated rosinate esters, such as dilinoleyl dimers of hydrogenated rosinate (Lusplan DD-DHR® or DD-DHR® from Nippon Fine Chemical),
and mixtures thereof.

Pulverulent Phase

As stated above, a powder composition according to the invention has a content of pulverulent phase of greater than or equal to 30% by weight, in particular greater than or equal to 40% by weight and more particularly ranging from 50% to 85% by weight relative to the total weight of said composition.

For the purposes of the present invention, this pulverulent phase is formed from any particulate solid material present in the composition and in particular from fillers and/or dyestuffs such as, for example, pigments.

Thus, the pulverulent phase comprises at least one filler and advantageously at least one dyestuff.

Fillers

The term “fillers” should be understood as meaning colourless or white, mineral or synthetic particles of any shape, which are insoluble and dispersed in the medium of the composition irrespective of the temperature at which the composition is manufactured.

Mineral or organic in nature, they make it possible to confer softness, mattness and uniformity of makeup on the composition.

The fillers used in the compositions according to the present invention may be of lamellar, globular or spherical form, or in any other form intermediate between these defined forms.

According to one particular embodiment, the composition of the invention comprises at least lamellar fillers and spherical fillers.

Among the mineral fillers that may be used in the compositions according to the invention, mention may be made of talc, mica, silica, magnesium aluminium silicate, trimethylsiloxysilicate, kaolin, calcium carbonate, magnesium hydrogen carbonate, hydroxyapatite, boron nitride, hollow silica microspheres (Silica Beads from Maprecos), glass or ceramic microcapsules, silica-based fillers, for instance Aerosil 200 or Aerosil 300; Sunsphere H-33 and Sunsphere H-51 sold by Asahi Glass; Chemicelen sold by Asahi Chemical; composites of silica and of titanium dioxide, for instance the TSG series sold by Nippon Sheet Glass, perlite powders and fluorphlogopite, and mixtures thereof.

Among the organic fillers that can be used in the compositions according to the invention, mention may be made of polyamide powders (Nylon® Orgasol from Atochem), poly-β-alanine powders and polyethylene powders, polytetrafluoroethylene powders (Teflon®), lauroyl lysine, starch, tetrafluoroethylene polymer powders, hollow polymer microspheres, for example comprising an (alkyl)acrylate, such as Expancel® (Nobel Industrie), elastomeric crosslinked organopolysiloxane spherical powders, described especially in document JP-A-02 243 612, such as those sold under the name Trefil Powder E-506C or DC9506 or DC 9701 by the company Dow Corning, silicone resins that are products of hydrolysis and polycondensation of mixtures of siloxanes of formulae (R)₃SiOHCH₃ and Si(OCH₃)₄, R representing an alkyl group containing from 1 to 6 carbon atoms (KSP100 from Shin Etsu for example), silicone resin microbeads (Tospearl® from Toshiba, for example), metal soaps derived from organic carboxylic acids containing from 8 to 22 carbon atoms, preferably from 12 to 18 carbon atoms, for example zinc stearate, magnesium stearate or lithium stearate, zinc laurate, magnesium myristate, the Polypore® L 200 (Chemdal Corporation), polyurethane powders, in particular powders of crosslinked polyurethane comprising a copolymer, said copolymer comprising trimethylol hexyllactone, for instance the hexamethylene diisocyanate/trimethylol hexyllactone polymer sold under the name Plastic Powder D-400® or Plastic Powder D-800® by the company TOSHIKI; carnauba microwaxes, such as the product sold under the name MicroCare 350® by the company Micro Powders, synthetic-wax microwaxes, such as the product sold under the name MicroEase 1145® by the company Micro Powders, microwaxes constituted of a mixture of carnauba wax and of polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 2505® by the company Micro Powders; and mixtures thereof.

Advantageously, the composition of the invention comprises at least spherical fillers capable of absorbing the oils.

In particular, mention may be made of:

silica microspheres, especially of open porosity or, preferably, hollow silica microspheres, such as the products Silica Beads SB 700/HA or Silica Beads SB 700 from the company Maprecos; these microspheres may be impregnated with a cosmetic active agent;

microporous polymer microspheres, which have a structure similar to that of a sponge; they generally have a specific surface area of at least 0.5 m²/g and in particular of at least 1 m²/g, said specific surface area having no upper limit other than that resulting from the practical possibility of making microspheres of very high porosity: the specific surface area may, for example, be up to 1000 m²/g or even more. Microspheres that may be mentioned include acrylic polymer microspheres, such as those made of crosslinked acrylate copolymer Polytrap 6603 Adsorber® from the company RP Scherer, and those made of polymethyl methacrylate Micropearl M 100® from the company SEPPIC;

polyurethane powder, such as the powdered copolymer of hexamethylene diisocyanate and of trimethylol hexyl lactone sold under the names Plastic Powder D-400 and T-7® by the company Toshiki;

elastomeric crosslinked organopolysiloxane spherical powders, described especially in document JP-A-02 243 612, such as those sold under the name DC 9506 Powder® by the company Dow Corning;

the carnauba wax microbeads sold under the name Microcare 350® by the company Micro Powders and the paraffin wax microbeads sold under the name Microease 114S® by the company Micro Powders;

micas or aluminosilicates of varied compositions that are especially in the form of flakes from 2 to 200 μm and preferably 5-70 μm in size and from 0.1 to 5 μm and preferably 0.2-3 μm in thickness, these micas possibly being of natural origin (for example muscovite, margarite, roscoelite, lepidolite or biotite) or of synthetic origin;

boron nitrides;

powders of spheronized, crosslinked or non-crosslinked synthetic polymers, for instance polyamide powders such as poly-β-alanine powder or Nylon powder, for example Orgasol powder from the company Atochem, polyacrylic acid or polymethacrylic acid powder, powders of polystyrene crosslinked with divinylbenzene, and silicone resin powders, and

bismuth oxychloride powders,

powders of organic materials of natural origin, for instance starches, especially corn starch, wheat starch or rice starch;

and mixtures thereof.

According to one particular and preferred embodiment, the composition comprises at least one lipophilic filler chosen from:
elastomeric crosslinked organopolysiloxane spherical powders, described especially in document JP-A-02 243 612, such as the products sold under the name DC 9506® or DC 9701® by the company Dow Corning, preferably DC 9506®;
hollow polymer microspheres, for example comprising an (alkyl) acrylate, such as Expancel® (Nobel Industrie),
the carnauba wax microbeads sold under the name Microcare 350® by the company Micro Powders and the paraffin wax microbeads sold under the name Microease 114S® by the company Micro Powders;
polyurethane powder, such as the powdered copolymer of hexamethylene diisocyanate and of trimethylol hexyl lactone sold under the names Plastic Powder D-400® and T-7® by the company Toshiki;
polyamide powders (Nylon® Orgasol from Atochem),
   and mixtures thereof.

According to one particularly preferred embodiment, the composition according to the invention comprises at least one filler chosen from crosslinked elastomeric organopolysiloxane spherical powders or else those with the INCI name “dimethicone/vinyl dimethicone crosspolymer”, described especially in document JP-A-02 243 612, such as those sold under the name DC9506® or DC9701 by the company Dow Corning, preferably DC9506 especially having an average particle size of 3 μm.

Advantageously, the elastomeric crosslinked organopolysiloxane in powder form is present in the composition in a content of active material ranging from 2% to 15% by weight and in particular from 3% to 10% by weight relative to the total weight of said composition.

According to one advantageous variant, a composition according to the invention comprises, as fillers, at least:
lamellar particles chosen from lamellar fillers and/or lamellar pearlescent agents, in particular chosen from boron nitride, mica, bismuth oxychloride and mixtures thereof, and
a crosslinked elastomeric organopolysiloxane spherical powder, in particular chosen from those with the INCI name “dimethicone/vinyl dimethicone crosspolymer”;
advantageously a polyurethane powder such as the powder of a copolymer of hexamethylene diisocyanate and of trimethylol hexyllactone; and/or
advantageously a polyamide (e.g. nylon) powder.

More specifically, a composition according to the invention may contain, as fillers, at least:
lamellar particles chosen from lamellar fillers and/or lamellar pearlescent agents, in particular chosen from boron nitride, mica, bismuth oxychloride and mixtures thereof, and
a crosslinked elastomeric organopolysiloxane spherical powder, in particular chosen from the products with the INCI name “dimethicone/vinyl dimethicone crosspolymer”, especially having an average particle size of 3 μm;
a polyurethane powder, such as the powdered copolymer of hexamethylene diisocyanate and of trimethylol hexyl lactone sold under the names Plastic Powder D-400® and T-7® by the company Toshiki; and
a polyamide (e.g. nylon) powder.

Advantageously, the fillers may be present in a hydrophobic coated form. They are more particularly fillers surface-treated with a hydrophobic agent to make them compatible with the fatty phase of the composition, especially so that they have good wettability with the oils of the fatty phase.

The hydrophobic treatment agent may be chosen from silicones such as methicones, dimethicones and perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, amino acids, N-acylamino acids or salts thereof; lecithin, isopropyl triisostearyl titanate, and mixtures thereof.

The N-acylamino acids may comprise an acyl group containing from 8 to 22 carbon atoms, for instance a 2-ethylhexanoyl, caproyl, lauroyl, myristoyl, palmitoyl, stearoyl or cocoyl group. The salts of these compounds may be the aluminium, magnesium, calcium, zirconium, zinc, sodium or potassium salts. The amino acid may be, for example, lysine, glutamic acid or alanine.

The term “alkyl” mentioned in the compounds cited above especially denotes an alkyl group containing from 1 to 30 carbon atoms and preferably containing from 5 to 16 carbon atoms.

According to one particular embodiment, the composition comprises at least one sericite (mica) treated with C₉-C₁₅ fluoroalcohol phosphates, as sold under the name PFX-5 Sericite FSE® by Daito Kasei Kogyo.

According to one preferred embodiment, the composition of the invention comprises, as fillers:
6 to 25% by weight of lamellar particles chosen from lamellar fillers and/or lamellar pearlescent agents, in particular chosen from boron nitride, mica, bismuth oxychloride and mixtures thereof,
2 to 12% by weight of a crosslinked elastomeric organopolysiloxane spherical powder, in particular chosen from those with the INCI name “dimethicone/vinyl dimethicone crosspolymer”, especially having an average particle size of 3 μm;
advantageously from 2% to 10% by weight of a polyurethane powder, such as the powdered copolymer of hexamethylene diisocyanate and of trimethylol hexyl lactone sold under the names Plastic Powder D-400 and T-7 by the company Toshiki; and
advantageously from 8% to 20% by weight of a polyamide (e.g. nylon) powder relative to the total weight of said composition.

Advantageously, a composition according to the invention may comprise a total content of fillers ranging from 10% to 60% by weight, preferably ranging from 12% to 50% by weight and preferentially ranging from 20% to 45% by weight, relative to the total weight of the composition.

As stated above, a composition according to the invention may also comprise, in its pulverulent phase, a colouring agent.

The colouring agent or pulverulent dyestuff according to the invention is preferably chosen from pigments, nacres, reflective particles, and mixtures thereof.

Pigments

The term “pigments” should be understood as meaning white or coloured, mineral or organic particles of any form, which are insoluble in the physiological medium and are intended to colour the composition.

The pigments may be white or coloured, and mineral and/or organic.

Among the mineral pigments that may be mentioned are titanium dioxide, optionally surface-treated, zirconium oxide or cerium oxide, and also zinc oxide, iron (black, yellow or red) oxide or chromium oxide, manganese violet, ultramarine blue, chromium hydrate and ferric blue, and metal powders, for instance aluminium powder and copper powder.

According to one preferred embodiment, the composition according to the invention comprises at least some iron oxides.

Advantageously, the pigments may be present in a hydrophobic coated form. They are more particularly pigments surface-treated with a hydrophobic agent especially as previously described in regard to the fillers. Examples of hydrophobic treated pigments that may be mentioned include the iron oxides and titanium dioxide coated with aluminium stearoyl glutamate sold under the commercial references NAI-TAO-77891®, NAI-C47-051-10®, NAI-C33-8001-10®, NAI-C33-7001-10®, or NAI-C33-9001-10® by the company Miyoshi Kasei.

The organic pigments may be chosen from the materials below, and mixtures thereof:
cochineal carmine,
organic pigments of azo dyes, anthraquinone dyes, indigoid dyes, xanthene dyes, pyrene dyes, quinoline dyes, triphenylmethane dyes and fluorane dyes.
   Among the organic pigments, mention may be made especially of the D&C certified pigments known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The chemical materials corresponding to each of the organic dyestuffs mentioned previously are mentioned in the publication “International Cosmetic Ingredient Dictionary and Handbook”, 1997 edition, pages 371 to 386 and 524 to 528, published by The Cosmetic, Toiletries and Fragrance Association, the content of which is incorporated into the present patent application by reference.

According to one preferred variant, a composition according to the invention may comprise a content of pigments ranging from 1% to 40% by weight, preferably from 5% to 30% by weight, in particular from 7% to 25% by weight and more particularly from 7% to 12% by weight, relative to the total weight of said composition.

The pulverulent phase according to the invention may also comprise nacres and reflective particles and mixtures thereof.

The term “nacre” should be understood as meaning coloured particles of any form, which may or may not be iridescent, especially produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, and nacreous pigments based on bismuth oxychloride. They may also be mica particles at the surface of which are superposed at least two successive layers of metal oxides and/or of organic dyestuffs. Mention may especially be made of synthetic mica coated with titanium dioxide and tin oxide, such as the product sold by the company Eckart under the name Syncristal Silver®.

The nacres may more particularly have a yellow, pink, red, bronze, orange, brown, gold and/or coppery colour or tint.

As illustrations of nacres that may be introduced into the composition, mention may be made of the gold-coloured nacres sold especially by the company Engelhard under the name Brillant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

Still as examples of nacres, mention may also be made of particles comprising a borosilicate substrate coated with titanium oxide.

Particles having a glass substrate coated with titanium oxide are especially sold under the name Metashine MC1080RY® by the company Toyal.

According to one particular variant, the compositions according to the invention may comprise from 0.1% to 40% by weight of nacres.

More preferably, they contain less than 7% by weight of nacres, especially from 0.1 to 7% by weight of nacres, in particular from 0.3 to 5% by weight of nacres.

Finally, the colouring agent according to the invention may be chosen from reflective particles.

The term “reflective particles” denotes particles whose size, structure, especially the thickness of the layer(s) of which they are made and their physical and chemical nature, and surface state allow them to reflect incident light. This reflection may, where appropriate, have an intensity sufficient to create at the surface of the composition or of the mixture, when it is applied to the support to be made up, points of overbrightness that are visible to the naked eye, i.e. more luminous points that contrast with their environment by appearing to sparkle.

The reflective particles may be selected so as not to significantly alter the colouration effect generated by the colouring agents with which they are combined, and more particularly so as to optimize this effect in terms of colour yield. They may more particularly have a yellow, pink, red, bronze, orange, brown, gold and/or coppery colour or tint.

These particles may have varied forms and may especially be in platelet or globular form, in particular in spherical form.

The reflective particles, whatever their form, may or may not have a multilayer structure and, in the case of a multilayer structure, may have, for example, at least one layer of uniform thickness, in particular of a reflective material.

When the reflective particles do not have a multilayer structure, they may be composed, for example, of metal oxides, especially titanium or iron oxides obtained synthetically.

When the reflective particles have a multilayer structure, they may comprise, for example, a natural or synthetic substrate, especially a synthetic substrate at least partially coated with at least one layer of a reflective material, especially of at least one metal or metallic material. The substrate may be made of one or more organic and/or inorganic materials.

More particularly, it may be chosen from glasses, ceramics, graphite, metal oxides, aluminas, silicas, silicates, especially aluminosilicates and borosilicates, and synthetic mica, and mixtures thereof, this list not being limiting.

The reflective material may comprise a layer of metal or of a metallic material.

Reflective particles are described especially in documents JP-A-09188830, JP-A-10158450, JP-A-10158541, JP-A-07258460 and JP-A-05017710.

Again as an example of reflective particles comprising a mineral substrate coated with a layer of metal, mention may also be made of particles comprising a silver-coated borosilicate substrate.

Particles having a glass substrate coated with silver, in the form of platelets, are sold under the name Microglass Metashine REFSX 2025 PS® by the company Toyal. Particles having a glass substrate coated with nickel/chromium/molybdenum alloy are sold under the name Crystal Star GF 550 and GF 2525® by this same company.

Use may also be made of particles comprising a metallic substrate such as silver, aluminium, iron, chromium, nickel, molybdenum, gold, copper, zinc, tin, manganese, steel, bronze or titanium, said substrate being coated with at least one layer of at least one metal oxide such as titanium oxide, aluminium oxide, iron oxide, cerium oxide, chromium oxide or silicon oxides, and mixtures thereof.

Examples that may be mentioned include aluminium powder, bronze powder or copper powder coated with SiO₂ sold under the name Visionaire by the company Eckart.

The reflective particles may be present in a content ranging from 0.1% to 40% by weight, in particular from 0.1% to 7% by weight, in particular from 0.3% to 5% by weight, relative to the total weight of said composition.

Thus, a composition according to the invention may comprise nacres/reflective particles in a content ranging from 0.1% to 40% by weight, especially from 0.1% to 7% by weight and in particular from 0.3% to 5% by weight relative to the total weight of said composition.

Preferably, the pulverulent phase comprises at least one compound chosen from:

organic pigments such as, for example:
the D&C pigments certified by the Food & Drug Administration as listed in the section “Color Additives—Batch Certified by the U.S. Food and Drug Administration” of the CTFA; mention may be made especially of Blue 1 and 4, Brown 1, Ext. Violet 2, Ext. Yellow 7, Green 3, 5, 6 and 8, Orange 4, 5, 10 and 11, Red 4, 6, 7, 17, 21, 22, 27, 28, 30, 36 and 40, Violet 2, Yellow 5, 6, 7, 8, 10 and 11,
the mineral pigments advantageously treated with a hydrophobic agent as described previously, such as titanium oxide, iron oxide, zirconium oxide, cerium oxide, iron oxide or chromium oxide, ferric blue, manganese violet, ultramarine blue, pink or violet, chromium hydrate, chromium hydroxide,
nacres, such as for example the mica or sericite coated with titanium oxide, the mica coated with titanium oxide and iron oxide, the synthetic mica coated with titanium oxide and tin oxide, the mica coated with amino acid, such as lauroyl lysine,
and mixtures thereof.

A composition according to the invention must be suitable for topical application to the skin or the lips and therefore generally comprises a physiologically acceptable medium, i.e. a medium compatible with the skin and/or the lips.

It is preferably a cosmetically acceptable medium, i.e. which has a pleasant colour, odour and feel and does not cause any unacceptable discomfort (stinging, tautness or redness) liable to discourage the consumer from using this composition.

The composition according to the invention may also comprise an aqueous phase. However, this aqueous phase must be used in an amount that is compatible with the pulverulent galenic form required according to the invention.

The water may be a floral water such as cornflower water and/or a mineral water such as Vittel water, Lucas water or La Roche Posay water and/or a spring water.

The aqueous phase may also comprise a polyol that is miscible with water at room temperature (25° C.) chosen especially from polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers; and mixtures thereof.

The composition according to the invention may comprise a polyol that is miscible with water at room temperature. A polyol that is suitable for use in the invention may be a compound of linear, branched or cyclic, saturated or unsaturated alkyl type, bearing on the alkyl chain at least two —OH functions and in particular at least three —OH functions. According to one preferred embodiment of the invention, said polyol is in liquid form at room temperature.

Advantageously, the polyol may be chosen, for example, from ethylene glycol, propylene glycol, 1,3-propanediol, butylene glycol, isoprene glycol, pentylene glycol, hexylene glycol, glycerol, polyglycerols, such as glycerol oligomers, for instance diglycerol, and mixtures thereof. According to a more particularly preferred embodiment of the invention, said polyol is glycerol.

In addition, the composition according to the invention may comprise a monoalcohol containing from 2 to 6 carbon atoms, such as ethanol or isopropanol.

Preferably, the composition according to the invention comprises less than 5% by weight and preferably less than 3% by weight of water relative to the total weight of the composition; preferably, the composition is free of water.

The composition may comprise other ingredients (adjuvants) usually used in cosmetics such as moisturizers, dyes, preferably liposoluble dyes, preserving agents, cosmetic or dermatological active agents, thickeners, surfactants, fragrances, and mixtures thereof.

According to one particular embodiment, the composition comprises at least one moisturizer (also known as a humectant).

The moisturizer(s) could be present in the composition in a content ranging from 0.1% to 15% by weight, especially from 0.5% to 10% by weight or even from 1% to 6% by weight, relative to the total weight of said composition.

Moisturizers or humectants that may especially be mentioned include sorbitol, polyhydric alcohols, preferably of C₂-C₈ and more preferably C₃-C₆, preferably such as glycerol, propylene glycol, 1,3-butylene glycol, pentylene glycol, hexylene glycol, dipropylene glycol, diethylene glycol and diglycerol, and a mixture thereof, glycerol and derivatives thereof, glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers, urea and derivatives thereof, especially Hydrovance (2-hydroxyethylurea) sold by National Starch, lactic acids, hyaluronic acid, AHAs, BHAs, sodium pidolate, xylitol, serine, sodium lactate, ectoin and derivatives thereof, chitosan and derivatives thereof, collagen, plankton, an extract of Imperata cylindra sold under the name Moist 24 by the company Sederma, acrylic acid homopolymers, for instance Lipidure-HM® from NOF Corporation, beta-glucan and in particular sodium carboxymethyl beta-glucan from Mibelle-AG-Biochemistry; a mixture of passionflower oil, apricot oil, corn oil and rice bran oil sold by Nestlé under the name NutraLipids®; a C-glycoside derivative such as those described in patent application WO 02/051 828 and in particular C-β-D-xylopyranoside-2-hydroxypropane in the form of a solution containing 30% by weight of active material in a water/propylene glycol mixture (60/40% by weight) such as the product manufactured by Chimex under the trade name Mexoryl SBB®; an oil of musk rose sold by Nestlé; spheres of collagen and of chondroitin sulphate of marine origin (Atelocollagen) sold by the company Engelhard Lyon under the name Marine Filling Spheres; hyaluronic acid spheres such as those sold by the company Engelhard Lyon; arginine, argan oil, and mixtures thereof.

Preferably, use will be made of a moisturizer chosen from glycerol, urea and derivatives thereof, especially Hydrovance® sold by National Starch, a C-glycoside derivative such as those described in patent application WO 02/051 828 and in particular C-β-D-xylopyranoside-2-hydroxypropane in the form of a solution containing 30% by weight of active material in a water/propylene glycol mixture (60/40% by weight) such as the product manufactured by Chimex under the trade name Mexoryl SBB®; argan oil, and mixtures thereof.

More preferably, glycerol will be used.

The liposoluble dyes are, for example, Sudan red, DC Red 17, DC Green 6, β-carotene, soybean oil, Sudan brown, DC Yellow 11, DC Violet 2, DC Orange 5 and quinoline yellow. The water-soluble dyes are, for example, beetroot juice and caramel.

In particular, use will be made of liposoluble dyes that can be conveyed in a liquid fatty phase.

Preparation Process

The compositions according to the invention may be prepared according to the following protocol.

The materials forming the pulverulent phase are introduced directly into a kneader-mixer usually used for pasty and/or powdery products, such as Baker-Perkins turbine mixers/granulators, dough mixers or continuous twin-screw kneaders of the BC21 or BC45 kneader-extruder type from the company Clextral, following in particular the suppliers' instructions.

When the composition comprises a non-emulsifying crosslinked elastomeric organopolysiloxane in powder form that is immiscible with the volatile oil, such as the product sold under the name 9506 Powder® from Dow Corning, it is added, preferably alone, to the mixer. The addition of the pulverulent phase and of the crosslinked elastomeric organopolysiloxane is preferably carried out at room temperature (20° C.).

The liquid fatty phase constituted completely or partly of non-volatile oil(s) is then added, with stirring, to the pulverulent phase until the two phases are homogenized.

The fatty powder obtained is then weighed in a small dish and is then subjected to a pressing operation, for example on a Vetraco machine: generally, a force of 200 to 2000 kg and preferentially 600 to 1000 kg is applied for a surface area of 63.48 cm², preferably a force of 800 kg for a surface are of 63.48 cm².

The powders according to the invention may be applied using a finger or advantageously using a specific applicator or sponge suitable for handling and applying a powder according to the invention to keratin materials, in particular the skin and the lips. By way of example, use could be made of a fingertip applicator such as that used in the examples below.

According to a first embodiment, the composition according to the invention is applied to the skin, in particular the skin of the face. It will especially be a foundation powder, a blusher or an eyeshadow.

According to another embodiment, the composition according to the invention is applied to the lips.

The invention is illustrated, in the example presented below, as a non-limiting illustration of the field of the invention.

Unless otherwise mentioned, the values in the example below are expressed as % by weight relative to the total weight of the composition.

### EXAMPLE 1

### Foundation Pressed Powders

| | FORMULA 1 | FORMULA 2 |
|---|---|---|
| | % by weight | % by weight |
| Powder phase | | |
| DIMETHICONE/VINYL DIMETHICONE | 8 | 3.94 |
| CROSSPOLYMER sold under the name | | |
| DOW CORNING 9506 POWDER ® by the company | | |
| DOW CORNING | | |
| BORON NITRIDE from Momentive Performance | 5.60 | 5.25 |
| Materials | | |
| MICA (and) C9-15 FLUOROALCOHOL PHOSPHATE | 10.90 | 8.09 |
| sold under the name PFX-5 SERICITE FSE ® by the | | |
| company DAITO KASEI KOGYO | | |
| MICROSPHERES (VINYLIDENE | 0.25 | 0.23 |
| CHLORIDE/ACRYLONITRILE/METHYL | | |
| METHACRYLATE COPOLYMER) EXPANDED BY | | |
| ISOBUTANE sold under the name EXPANCEL 551 DE | | |
| 40 D42 ® by AKZO NOBEL | | |
| Carnauba wax microbeads sold under the name | 2.28 | 2.10 |
| MICROCARE 350 ® by Micro Powders | | |
| TITANIUM DIOXIDE (and) LAUROYL LYSINE sold | 6.83 | 6.30 |
| under the name LL 5 TITANIUM DIOXIDE CR 50 by | | |
| DAITO KASEI KOGYO | | |
| RONAFLAIR LF 2000 BISMUTH OXYCHLORIDE | 7.97 | 7.35 |
| from the company MERCK | | |
| NYLON-12 sold by ARKEMA | 9.26 | 8.54 |
| HDI-TRIMETHYLOL HEXYL LACTONE | 3.99 | 3.68 |
| CROSSPOLYMER sold under the name PLASTIC | | |
| POWDER D 400 ® by TOSHIKI PIGMENT | | |
| red, yellow and black IRON OXIDES | 1.23 | 3.11 |
| NACRES | — | 2.63 |
| Binder phase | | |
| ISONONYL ISONONANOATE | 3.15 | 3.48 |
| POLYDIMETHYLSILOXANE having a viscosity of 5 cst | qs 100 | qs 100 |
| at 25° C. sold under the name XIAMETER PMX-200 | | |
| SILICONE FLUID 5CS ® by DOW CORNING | | |
| NYLON-611/DIMETHICONE COPOLYMER sold | 0.90 | 0.99 |
| under the name DOW CORNING 2-8179 GELLANT ® | | |
| by DOW CORNING | | |
| CAPRYLYL GLYCOL | 0.50 | 0.55 |
| GLYCEROL | — | 1.43 |
| HYDROXYETHYL UREA from NATIONAL STARCH | — | 1.43 |
| C-β-D-xylopyranoside-2-hydroxy-propane in the form of | — | 0.33 |
| a solution containing 30% by weight of active material in | | |
| a water/propylene glycol mixture (60/40% by weight) | | |
| under the trade name MEXORYL SBB ® sold by | | |
| CHIMEX | | |
| ARGANIA SPINOSA KERNEL OIL | — | 0.24 |

The two compositions are prepared according to the following protocol:
1) Preparation of the powder phase: All the pulverulent raw materials are combined in the bowl of a Baker mixer. The blade is programmed at 3000 rpm and the lump breaker at 2700 rpm. The mixing is carried out for 20 minutes.
2) Preparation of the binder phase: the binder phase is homogenized with a rotor-stator mixer at 2000 rpm for 10 minutes.
3) Manufacture of the cosmetic product: in a dough mixer equipped with a pastry-type scraping blade, the speed of which is 2 revolutions/second. The powder is placed in the bowl, and the binder phase is added thereto at a flow rate of 10 g/min.
4) The preparation thus obtained is then pressed at 10 kg/cm².

The compositions thus prepared have an average hardness of 0.1 N measured according to the protocol described previously in the description.

The two compositions are tested by 10 regular users of compact powders, all skin types except extreme types, from 25 to 50 years old.

Each of the powders is applied to the face by each of the users with the aid of a fingertip applicator.

The texture surprises the users. Indeed, the very compact and dense aspect of the material is visually similar to a creamy texture, reminding them of the appearance of a cream compact foundation. However, to the touch, against all expectation, the women have the pleasant perception of the product's non-powdery and unctuous character. Unlike their customary powder, they particularly like its non-volatile aspect during take up and on application.

They are won over by the feeling of softness that it imparts.

The powder adheres well to the fingertip applicator and the application proves to be easy.

The powder makes it possible to mattify the skin and to diminish slight colour imperfections. It unifies the complexion while leaving a thin film that gives a natural look to the skin.

The users are satisfied with the sensation of comfort that they feel after application and also the feeling of lightness.

### EXAMPLE 2

### Foundation Loose Powder

| | % by weight |
|---|---|
| Powder phase | |
| DIMETHICONE/VINYL DIMETHICONE | 11.6 |
| CROSSPOLYMER sold under the name | |
| DOW CORNING 9506 POWDER from DOW | |
| CORNING | |
| BORON NITRIDE | 7.86 |
| MICA (and) C9-15 FLUOROALCOHOL | 15.4 |
| PHOSPHATE sold under the name PFX-5 | |
| SERICITE FSE ® by the company DAITO KASEI | |
| KOGYO | |
| MICROSPHERES (VINYLIDENE | 0.4 |
| CHLORIDE/ACRYLONITRILE/METHYL | |
| METHACRYLATE COPOLYMER) EXPANDED | |
| BY ISOBUTANE sold under the name EXPANCEL | |
| 551 DE 40 D42 ® by AKZO NOBEL | |
| Carnauba wax microbeads sold under the name | 3.3 |
| MICROCARE 350 ® by Micro Powders | |
| TITANIUM DIOXIDE (and) LAUROYL LYSINE | 9.7 |
| sold under the name LL 5 TITANIUM DIOXIDE | |
| CR 50 ® by DAITO KASEI KOGYO | |
| RONAFLAIR LF 2000 ® BISMUTH | 11.35 |
| OXYCHLORIDE from the company MERCK | |
| NYLON-12 sold by ARKEMA | 13 |
| HDI-TRIMETHYLOL HEXYL LACTONE | 5.6 |
| CROSSPOLYMER sold under the name PLASTIC | |
| POWDER D 400 ® by TOSHIKI PIGMENT | |
| red, yellow and black IRON OXIDES | 1.79 |
| Binder phase | |
| ISONONYL ISONONANOATE | 1.45 |
| POLYDIMETHYLSILOXANE having a viscosity of | qs 100 |
| 5 cst at 25° C. sold under the name XIAMETER | |
| PMX-200 SILICONE FLUID 5CS ® by DOW | |
| CORNING | |
| NYLON-611/DIMETHICONE COPOLYMER sold | 0.42 |
| under the name DOW CORNING 2-8179 | |
| GELLANT by DOW CORNING | |
| CAPRYLYL GLYCOL | 0.23 |

This loose powder is prepared according to the protocol described in Example 1, following steps 1) to 3). It is, on the other hand, screened and therefore not pressed.

A soft and light powder is obtained which mattifies the skin and unifies the complexion.

1-15. (canceled)

16. Solid cosmetic makeup and/or care composition comprising:
at least 30% by weight of a pulverulent phase relative to its total weight, and comprising as filler, at least one crosslinked elastomeric organopolysiloxane spherical powder;
at least 15% by weight of non-volatile oil(s) relative to its total weight, and
at least one dyestuff;
said composition being in the form of a powder.

17. Solid cosmetic makeup and/or care composition comprising:
at least 30% by weight of a pulverulent phase relative to its total weight; and
at least 15% by weight of non-volatile oil(s) relative to its total weight, comprising silicon oils chosen from cyclic non-volatile polydimethylsiloxanes and phenyl silicones;
said composition being in the form of a powder.

18. Solid cosmetic makeup and/or care composition comprising:
at least 30% by weight of a pulverulent phase relative to its total weight; and
at least 15% by weight of non-volatile oil(s) relative to its total weight, and comprising as non-volatile oil(s), at least one isononanoic acid ester,
said composition being in the form of a powder.

19. Composition according to claim 18, wherein it comprises as non-volatile oil(s), at least one isononyl isononanoate.

20. Solid cosmetic makeup and/or care composition comprising:
at least 30% by weight of a pulverulent phase relative to its total weight; and
at least 15% by weight of non-volatile oil(s) relative to its total weight, and comprising as non-volatile oil(s), at least one non-volatile hydrocarbon-based oil chosen from the oils of formula R₁COOR₂ chosen from C₁₂-C₁₅ alcohol benzoates,
said composition being in the form of a powder.

21. Solid cosmetic makeup and/or care composition comprising:
at least 30% by weight of a pulverulent phase relative to its total weight,
at least 15% by weight of non-volatile oil(s) relative to its total weight, and
at least a silicone resin chosen from siloxysilicate resins,
said composition being in the form of a powder.

22. Solid cosmetic makeup and/or care composition comprising:
at least 30% by weight of a pulverulent phase relative to its total weight,
a total filler content ranging from 10% to 60% by weight relative to its total weight,
a content of non-volatile oil(s) ranging from 30% to 45% by weight relative to its total weight, and comprising as non-volatile oil(s), at least one non-volatile hydrocarbon-based oil,
said composition being in the form of a powder.

23. Composition according to claim 16, wherein it is a pressed powder.

24. Composition according to claim 23, wherein it has a hardness that varies from 0.01 to 5 newtons measured according to the protocol described in the description.

25. Composition according to claim 16, wherein it has a content of pulverulent phase of greater than or equal to 40% by weight.

26. Composition according to claim 16, wherein the pulverulent phase comprises at least one filler and at least one dyestuff.

27. Composition according to claim 16, wherein it comprises a total filler content ranging from 10% to 60% by weight, relative to the total weight of the composition.

28. Composition according to claim 16, wherein it comprises a pigment content ranging from 1% to 40% by weight.

29. Composition according to claim 16, wherein it comprises nacres/reflective particles in a content ranging from 0.1% to 40% by weight.

30. Composition according to claim 16, wherein it comprises a content of non-volatile oil(s) ranging from 20% to 100% by weight, relative to the total weight of the liquid fatty phase.

31. Composition according to claim 16, wherein it comprises at least one non-volatile silicone oil.

32. Composition according to claim 31, wherein the non-volatile silicone oil(s) represent(s) more than 50% by weight of the total non-volatile oils or even more than 90% by weight of the total non-volatile oils.

33. Composition according to claim 16, wherein it also comprises at least one crosslinked elastomeric organopolysiloxane powder.

34. Composition according to claim 16, wherein it comprises, as fillers, at least
lamellar particles chosen from lamellar fillers and/or lamellar pearlescent agents, and
a crosslinked elastomeric organopolysiloxane spherical powder;
advantageously a polyurethane powder such as the powder of a copolymer of hexamethylene diisocyanate and of trimethylol hexyllactone; and/or
advantageously a polyamide (e.g. nylon) powder.

35. Composition according to claim 16, wherein it also comprises at least one moisturizer.

36. Method for making up and/or caring for keratin materials in which a composition according to claim 16 is applied to said keratin materials.
